(19) United States
(12) Patent Application Publication
(10) Pub. No.: US 2009/0280196 A1
(43) Pub. Date: Nov. 12, 2009

(54) CISSUS QUADRANGULARIS PLANT EXTRACTS FOR TREATING OSTEOPOROSIS AND THE EXTRACTION PROCESS THEREOF

(76) Inventor: Villoo Morawala Patell, Karnataka (IN)

Correspondence Address: Pearl Cohen Zedek Latzer, LLP, 1500 Broadway, 12th Floor, New York, NY, 10036, US

(21) Appl. No.: 12/307,776

(22) PCT Filed: Jul. 9, 2007
(86) PCT No.: PCT/IB07/02066

§ 371 Date: Jan. 7, 2009

(30) Foreign Application Priority Data
Jul. 7, 2006 (IN) 119/CHE/2006

(51) Int. Cl. A61K 36/00

(52) U.S. Cl. 424/725

(57) ABSTRACT

The invention relates to compositions and methods for preventing, treating, or managing osteoporosis or conditions which are characterized by increased bone resorption, comprising administration of a prophylactically and therapeutically effective amount of Cissus quadrangularis plant or extracts thereof to a mammal in need of such therapy. Preferably the mammal is human and the compositions comprise of single extract or a combination of extracts thereof. The present invention further relates to extracts which are isolated from different parts of Cissus quadrangularis plant, the preparation of such extracts, the medicaments containing said extracts, and the use of these extracts and constituents for the preparation of a medicament. The present invention also relates to the process for preparing the extracts from various parts of Cissus quadrangularis plant.

### FIELD OF INVENTION

The invention relates to compositions and methods for preventing, treating, or managing osteoporosis or other related disorders such as bone loss, bone fracture, glucocorticoid induced osteoporosis, Pagets disease, osteoarthritis, peri-prosthetic osteolysis, cartilage degeneration, osteogenesis imperfecta and the like, comprising administration of a prophylactically and therapeutically effective amount of Cissus quadrangularis plant or extracts thereof to a mammal in need of such therapy. Preferably the mammal is human and the compositions comprise of single extract or a combination of extracts thereof.

The present invention further relates to extracts which are isolated from different parts of Cissus quadrangularis plant, the preparation of such extracts, the medicaments containing said extracts, and the use of these extracts and constituents for the preparation of a medicament.

The present invention also relates to the process for preparing the extracts from the whole plant of Cissus quadrangularis plant.

### BACKGROUND OF INVENTION

Osteoporosis is a skeletal disorder that is characterized by low bone mass and micro-architectural deterioration of bone tissue. Affecting 200 million individuals world-wide, osteoporosis is the most common metabolic bone disorder which leads to an increased level of bone fragility and susceptibility to fracture (Walker-bone, et al. 2002; Lin and Lane, 2004).

### A. Classification of Osteoporosis

Based on its etiology, osteoporosis is categorized as a primary or secondary disease. The latter involves the onset of osteoporosis as a result of an existing condition such as an endocrine disorder, the use of certain medications, a hematopoietic disorder, immobilization, or a nutritional, gastrointestinal or connective tissue disorder (Lin and Lane, 2004). Primary osteoporosis is further subdivided into two types. Type I generally occurs in postmenopausal women and is attributed to loss of gonadal hormone function, such as estrogen deficiency associated with menopause (Lin and Lane, 2004; Simon, 2004). Type II osteoporosis generally called as senile osteoporosis is age-related, affecting both men and women over the age of 60. (Lin and Lane 2004) Through the assessment of bone mineral density (BMD; in g/cm 2) using dual energy x-ray absorptiometry (DEXA), the World Health Organization has defined osteoporosis as a BMD more than 2.5 standard deviations below the mean of normal, healthy individuals at their peak bone mass (Lin and Lane, 2004; Simon, 2004; Christodoulou and Cooper, 2003).

### B. Normal and Abnormal Bone Remodeling

Through regulation by sywhole plantic hormones, including vitamin D, growth hormone (GH) and parathyroid hormone (PTH), as well as local signaling through cytokines and growth factors, bone undergoes continuous remodeling even after complete skeletal growth has been attained. Bone remodeling occurs at specific sites on the bone surface known as basic multicellular units and is carried out by osteoclasts (bone resorption cells) and osteoblasts (bone formation cells) (Tolstoi 2004, Manolagas and Weinstein, 1999). Both osteoblasts and osteoclasts are derived from precursors originating in the bone marrow and the formation and activation of these cells is regulated by cytokines and growth factors also produced in the bone marrow, which are in turn controlled by sywhole plantic factors and mechanical stimuli (Manolagas and Weinstein 1999). The bone remodeling cycle is highly dependent on a delicate balance between regulatory signaling and cellular activity. Loss of the capacity to recruit active osteoblasts or deactivate osteoclasts results in a net bone loss and can lead to the onset of osteoporosis.

### III. The Etiology of Osteoporosis

Although numerous risk factors have been identified to increase the likelihood of developing this disease, including Caucasian race, advanced age, female gender, history of fracture, smoking and alcoholism, the exact cause of osteoporosis has not yet been identified. Despite this, numerous theories have been proposed in an attempt to explain its etiology. Some theories regarding the etiology of osteoarthritis include bone cell senescence, lifestyle factors (primarily exercise and nutrition) and loss of vitamin D metabolism with age (Tsai, et al., 1984). The latter hypothesis infers that aging leads to an impaired metabolism of vitamin D. Activated vitamin D is a signaling molecule that is largely involved in the regulation of intestinal calcium absorption Tsai, et al., 1984). Therefore, poor vitamin D metabolism leads to a decrease in intestinal calcium absorption and results in PTH signaling, by the endocrine sywhole plant, to withdraw calcium from the bones. Over time, this continuous removal of calcium from the bones leads to decreased bone mass and development of osteoporosis.

### IV. Treatment

There are different types of drugs used to treat osteoporosis: antiresorptive drugs, which slow the progressive thinning of bone, bone forming drugs which help to rebuild the skeleton, and drugs with a more complex mechanism of action. In addition to drug therapy, calcium and vitamin D supplements might also be prescribed to ensure adequate intake and to ensure maximum effectiveness of the drug therapy.

Bisphosphonates inhibit bone resorption. They are currently the first choice of treatment in a variety of bone metabolism disorders characterised by high bone resorption.

Selective estrogen receptor modulators (SERMS) mimic estrogens in some tissues and anti-estrogens in others, and ideally provide the bone-retaining effects of estrogen without its unwanted side effects. Currently, the only marketed SERM is raloxifene. Raloxifene prevents bone loss and is indicated for the prevention and treatment of vertebral fractures in postmenopausal women.

A new drug has been approved for the treatment of osteoporosis—this time one that improves bone formation, as opposed to the action of available drugs that is usually the prevention or slowing bone resorption. It is teriparatide, a 34-amino-acid polypeptide produced by a recombinant DNA technique, which represents the biologically active part of human parathyroid hormone. It has to be given once daily by subcutaneous injection. They treat osteoporosis by stimulating bone-forming cells called osteoblasts. It has a dramatic effect on bone, increasing bone mineral density in the spine by as much as 13% in 18 months and reducing the risk of fracture by as much as 90%. The reason that patients are not using this drug is “cost”. Forteo costs about $600 a month, and it also must be injected every day. For those reasons, it's generally only prescribed for patients with severe osteoporosis, or who have already had one or more fractures.

A nutritional approach would be an inexpensive means to achieve this goal. However, the effects of the nutritional strategies recommended today are rather modest. Thus, research into novel nutritional strategies preventing bone loss and improving bone formation is needed.

Cissus quadrangularis is an indigenous medicinal plant of India. The use of this plant by the common folk for promoting fracture-healing process is an old practice. It has been prescribed in ancient Ayurvedic texts by Bhava Prakash and Chakra Dutta as a general tonic especially for the fractured patient. Since then it has been in extensive use by bonesetters both for external application and as an internal medicine to be taken with milk. The stem of Cissus quadrangularis is also reputed in Ayurveda as alternative, anthelmintic, dyspeptic, digestive, tonic, analgesic in eye and ear diseases, in the treatment of irregular menstruation and asthma, and in complaints of the back and spine. Scientific studies have revealed the Cissus extract to possess cardiotonic and androgenic property.

Cissus quadraingularis contains high amount of vitamin C, carotene A, anabolic steroidal substances and calcium. These anabolic steroidal principles from Cissus quadrangularis showed a marked influence in the rate of fracture-healing by influencing early regeneration of all connective tissues of mesenchyma origin, namely the fibroblasts, the chondroblasts and osteoblasts involved in the healing and quicker mineralization of the callus. It has greater impact on osteoblastic proliferation than other cellular responses.

Cissus quadrangularis causes less amount of tissue reaction in the fractured region leading to optimum decalcification in the early stage with minimum of callus formation. Hence deposition of calcium is just enough to join the two broken segments of bone so that it's remodeling takes much faster. This early completion of calcification process and earlier remodeling phenomenon lead to early recovery of Cissus treated animals. Cissus is also shown to cause early gain in the tensile strength of fractured bones of about 90 percent of its normal strength at the end of 6 week. Cissus quadrangularis builds up the chemical composition of the fractured bone namely its mucopolysaccharides, collagen, calcium, phosphorus and others as well as its functional efficiency. Mucopolysaccharides play an important role in the healing by supplying raw materials for repairs. Therefore it seems that in the early period of bone fracture healing the greater the accumulation of these materials more rapid will be the rate of healing. Rapid the utilization of these raw materials earlier will be completion of healing process. Cissus quadrangualris not only causes the greater accumulation of mucopolysaccharides but also an earlier disappearance of mucopolysaccharides from the fractured area, associated with the earlier calcification and firmer callus formation.

In clinical trials Cissus quadrangularis as per radiological and clinical observations has been found to cause considerable reduction in the healing time of fractures by 55-33 percent. In few of the treated cases, although radiologically only, an early callus formation was observed but clinically the symptoms of fracture such as pain, tenderness, and swelling were significantly absent.

Cissus quadrangularis is shown to neutralize the anti-anabolic effect of steroids like cortisone in healing of fractures. Antianabolic effects of cortisone include inhibition of tissue regeneration and repair, also retarding formation of the specific skeletal structures. In such conditions even if the cartilage tissue is produced, its maturation and ultimate bone replacement do not take place in the normal pattern. It has main inhibitory action on fibroblasts and mast cells, which produce mucopolysaccharides of connective tissue. There have been reports that continuous intake of corticosteroids induces osteoporosis and pseudofractures in the bone.

Imbalance in the activities of osteoclasts (cells responsible for bone loss) and osteoblasts (cells responsible for bone formation) may lead to osteoporosis and fractures in postmenopausal women. In osteoporosis, the bones begin to deteriorate due to calcium deficiency as a result of the body's efficiency in maintaining mineral balance in the blood at the expense of bone integrity. During menopause the decrease in hormones affects the body's ability to maintain calcium levels resulting in an increased loss of minerals from the bones. Postmenopausal women are at particular risk to osteoporosis because the loss of estrogen associated with the menopause leads to bone loss of much greater magnitude than one would expect on the basis of age alone. Cissus quadrangularis with significant ability to inhibit antianabolic effects and bone fracture healing effects is likely to exert some beneficial effects on recovery of bone mineral density in postmenopausal osteoporosis.

In the light of its traditional use in bone healing and scientific evidence, this species appears to be a promising candidate for the antiosteoporotic activity.

### SUMMARY OF THE INVENTION

The invention relates to compositions and methods for preventing, treating, or managing osteoporosis other related disorders such as bone loss, bone fracture, glucocorticoid induced osteoporosis, Pagets disease, osteoarthritis, peri-prosthetic osteolysis, cartilage degeneration, osteogenesis imperfecta and the like, comprising administration of a prophylactically and therapeutically effective amount of Cissus quadrangularis plant or extracts thereof to a mammal in need of such therapy. Preferably the mammal is human and the compositions comprise of single extract or a combination of extracts thereof.

The present invention further relates to extracts which are isolated from whole plants of Cissus quadrangularis plant, the preparation of such extracts, the medicaments containing said extracts, and the use of these extracts and constituents for the preparation of a medicament.

The present invention also relates to the process for preparing the extracts from Cissus quadrangularis plant whole plants. The process comprises of:
Cleaning the Cissus quadrangularis plant material.
Pulverizing Cissus quadrangularis plant material to a powder by feeding on to a hammer mill or like apparatus at room temperature and passing through the mesh to get uniform size of powder.
Perform Direct/Successive extraction of plant parts in soxhlet apparatus or in flask at room temperature or at higher temperature with polar and non-polar solvent(s).
Lyophilize the extracts dry, under vacuum.
The lyophilized extracts are further taken for cell based and cell free bioassay.

The compositions/medicaments may contain a pharmaceutically acceptable carrier, excipient, or diluent. The compositions can be included as unit dosage suitable for parenteral, oral, or intravenous administration to a human. Alternatively, the compositions are dietary supplements, food compositions or beverage compositions suitable for human or animal consumption.

The invention further describes the biotherapeutic potential of various extracts of Cissus quadrangularis as described above, by studying their performance in cell based assay models.

These and other objects of the invention will be more fully understood from the following description of the invention, the referenced drawings attached hereto and the claims appended hereto.

### BRIEF DESCRIPTION OF FIGURES

FIG. 1: The cell viability for the extracts AN01, AN02, AN03

FIG. 2 The cell viability for the extracts AN 19

FIG. 3 The CTX release for plant extract AN01, AN02, AN03 for the resorption experiment

FIG. 4 The CTX release for plant extract AN19 for the resorption experiment

FIG. 5 The cell viability for plant extract AN01, AN02, AN03 for the resorption experiment

FIG. 6 The cell viability for plant extra AN19 for the resorption experiment

FIG. 7 The CTX release for plant extract AN01 lead characterization

FIG. 8 The CTX release for plant extract AN02 lead characterization

FIG. 9 The CTX release for plant extract AN03 lead characterization

FIG. 10 The cell viability for plant extract AN01 lead characterization

FIG. 11 The cell viability for plant extract AN02 lead characterization

FIG. 12 The cell viability for plant extract AN03 lead characterization

FIG. 13 The TRAP activity for plant extract AN01 lead characterization

FIG. 14 The TRAP activity for plant extract AN02 lead characterization

FIG. 15 The TRAP activity for plant extract AN03 lead characterization

FIG. 16 The ALP activity for plant extract AN01 for the bone formation experiment

FIG. 17 The ALP activity for plant extract AN19 for the bone formation experiment

FIG. 18 The cell viability for plant extracts AN01, AN03 for the bone formation experiment

FIG. 19 The cell viability for plant extracts AN19 for the bone formation experiment

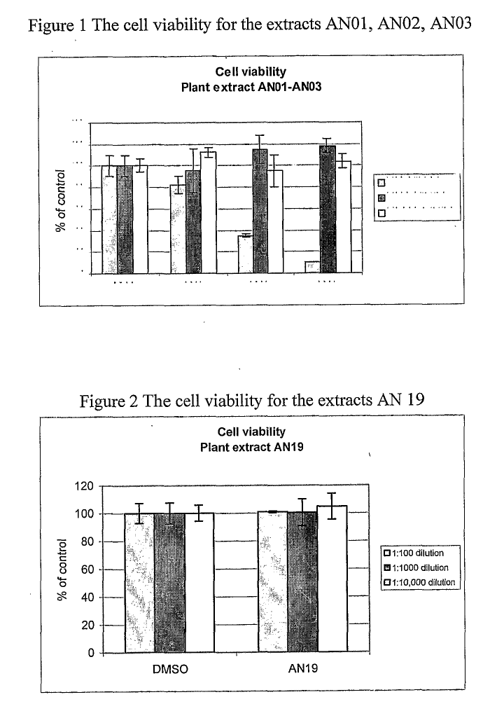

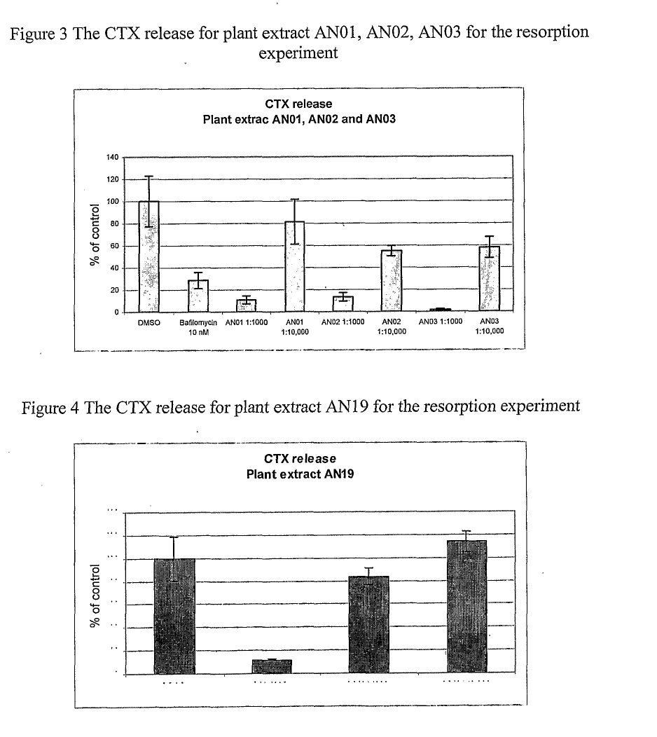

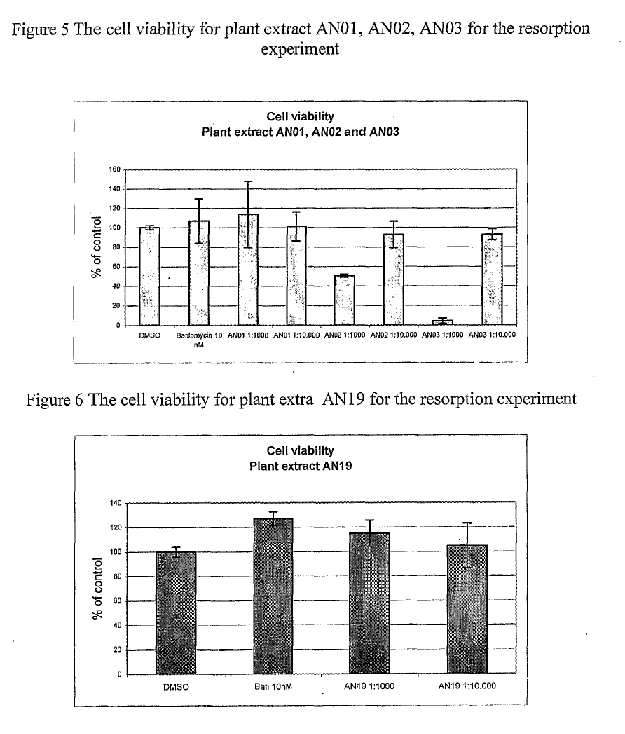

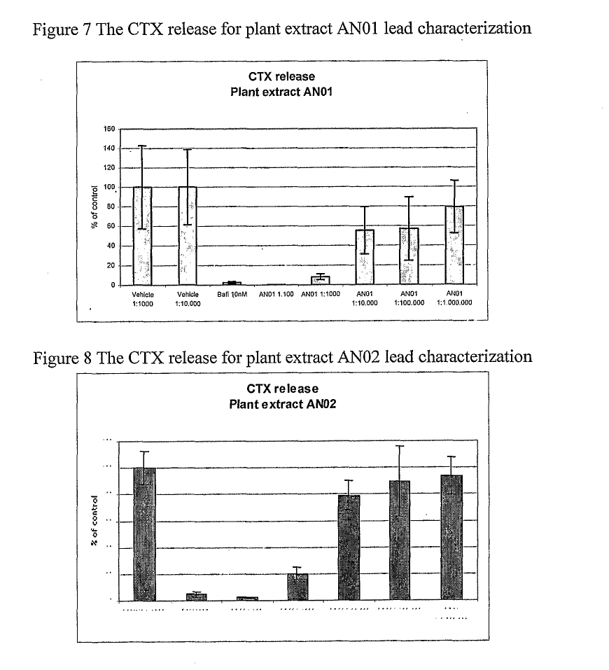

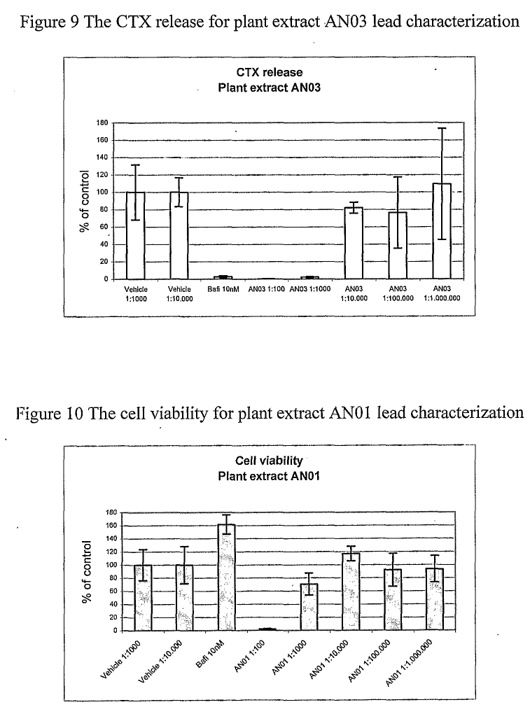

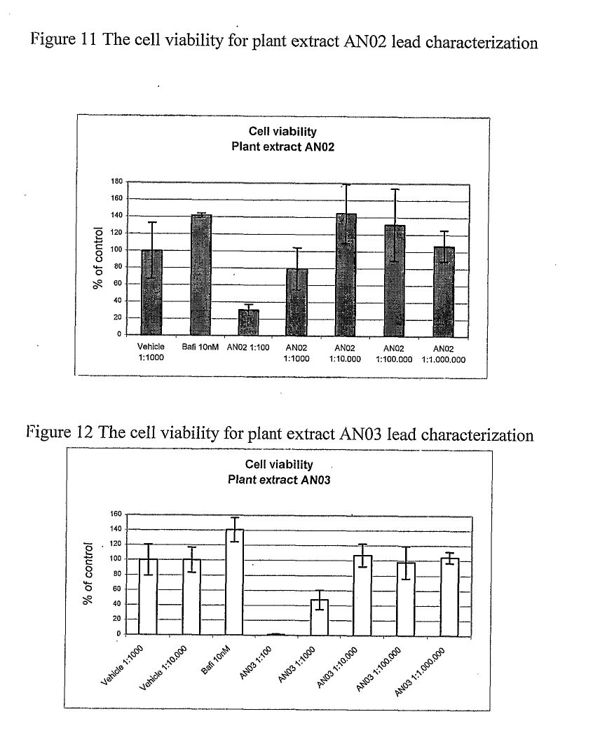

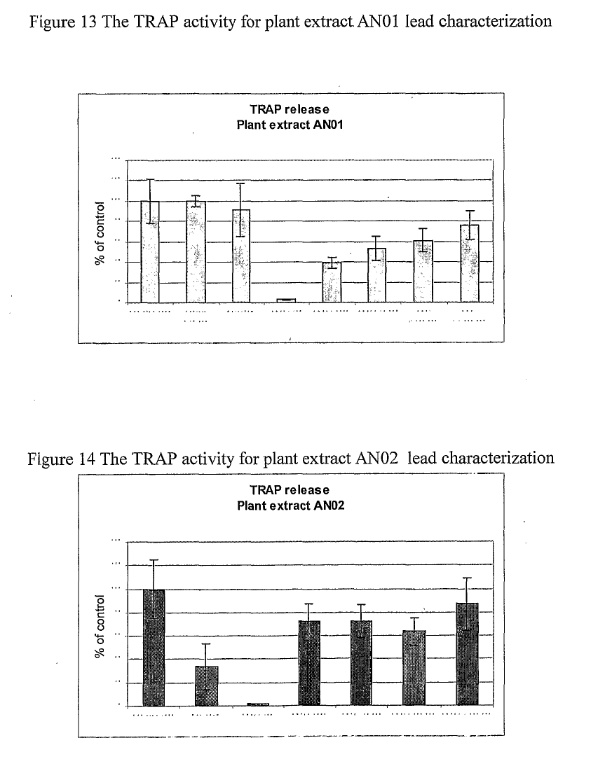

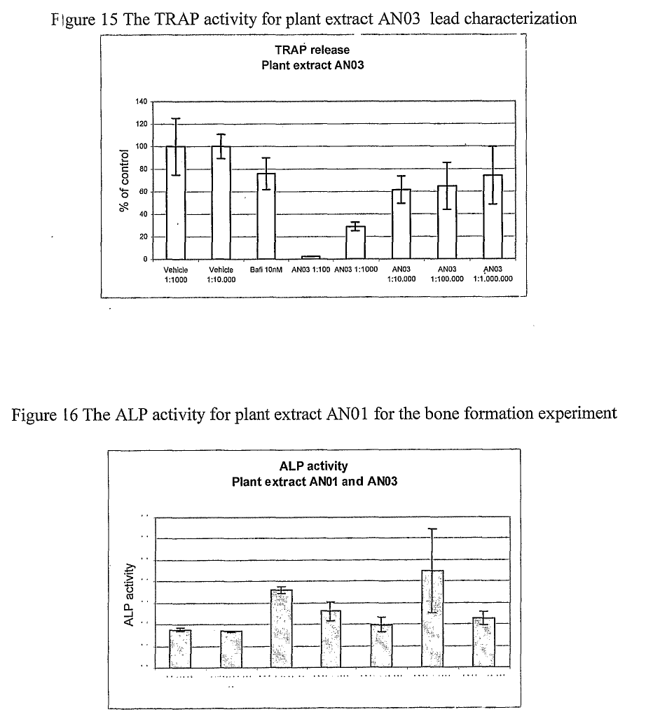

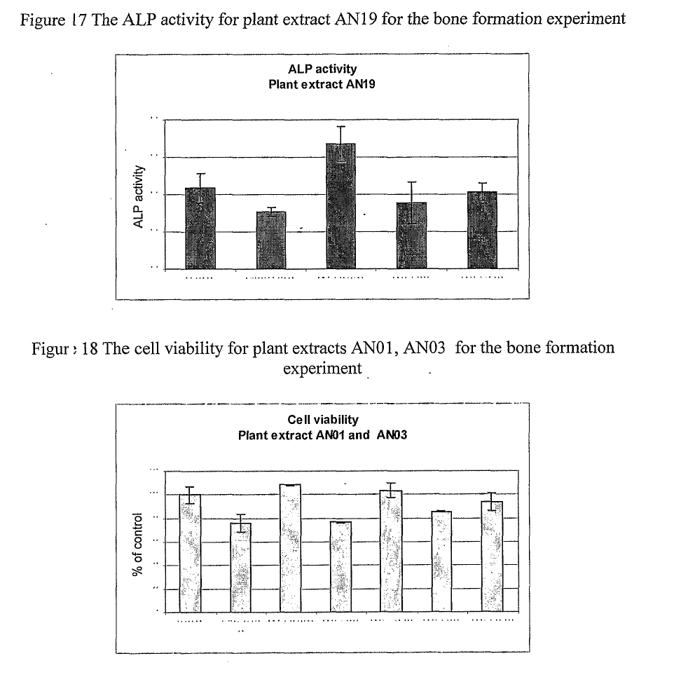

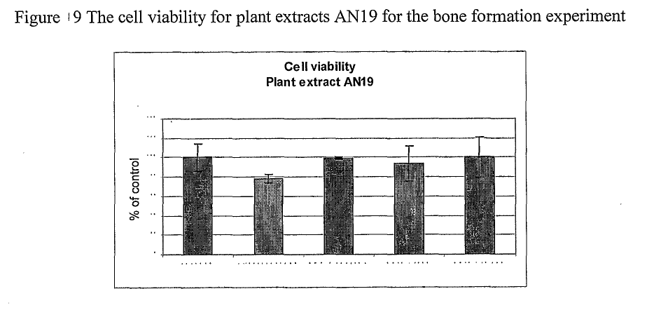

### DETAILED DESCRIPTION OF THE INVENTION

In a first aspect of the invention there is provided a method for treating a disease in a mammal, which comprises administering to the said mammal an effective non-toxic amount of at least an extract from Cissus quadrangularis as defined herein. Preferably the mammal is a human being. The skilled addressee will appreciate that “treating a disease” in a mammal means treating, that is to say, alleviating symptoms of the disease and may also mean managing a disease in the sense of preventing such a disease state either advancing ie getting worse or becoming more invasive, or slowing down the rate of advance of a disease.

In a second aspect of the invention, there is a provided a prophylactic method for preventing the occurrence of a disease state in a mammal which comprises administering to the said mammal an effective non-toxic amount of an extract from Cissus quadrangularis as defined herein in the preparation of a comestible (=foodstuff) for prophylaxis against the occurrence of Osteoporosis. Preferably the mammal is human and the said extract comprises a single extract from a plant part of Cissus quadrangularis or a combination of extracts therefrom as detailed herein. Thus the present invention further relates to extracts, which may be isolated from whole plants of the Cissus quadrangularis plant, the preparation of such extracts, medicaments comprising such extracts, and the use of these extracts and constituents for the preparation of a medicament.

Extracts of the present invention can be isolated from whole plants of Cissus quadrangularis, using conventional organic solvent extraction and supercritical fluid extraction technology. Generally, extracts of the invention capable of functioning in a prophylactic or therapeutic manner as outlined herein can be extracted from any Cissus quadrangularis plant, depending on the end purpose that is required of the extract.

In a third aspect of the present invention there is provided a process for preparing extracts of the invention from plant parts of Cissus quadrangularis that comprises:
Pulverizing selected plant material to a powder;
Subjecting the powdered plant material to solvent extraction;
Lyophilizing the obtained extracts.

The choice of selected plant material may be of any type but is preferably the whole plants of the Cissus quadrangularis plant.

The solvent extraction process may be selected from direct types such as extraction from plant parts in soxhlet apparatus or in flasks at room temperature or at higher temperature with polar and/or non-polar solvent(s). Typically, the extraction process is as outlined herein.

It will be apparent to the skilled addressee that the selection of solvent, or mixtures of solvents for each step in the isolation of extracts of the invention showing activity can be guided by results of bioassay analysis of separate fractions, for procedure as indicated herein and/or as shown in the procedures.

### Cell Based Toxicity Tests:

In view of the important role played by osteoblasts in regulating growth and in bone remodelling, a series of tests were conducted to evaluate whether the presence of phytoextracts obtained would influence the growth of osteoblastic cells in vitro.

In one embodiment of the present invention study of the in-vitro toxicity was undertaken through a series of tests that are conducted to evaluate the effect of the extracts on the growth and viability of the osteoblastic cells. To this end, mouse osteoblastic cells, MC3T3 (mouse clavarial osteoblast like cells) were seeded at a density of 20,000 cells in 96 well plates and cultured for one day prior to the addition of the plant extracts. The stock solutions of the extracts dissolved in DMSO were diluted to 3 different concentrations. The cells after addition of the extracts are cultured for 3 days and cell viability is measured by a colorimetric based cell viability assay as exemplified in the detailed description. Alternatively, as will be readily apparent to one skilled in the art separate cytotoxicity tests, stability tests and the like can be conducted to evaluate the toxicity of the extracts or compositions can be conducted.

Furthermore, also as readily apparent to one skilled in the art, the therapeutic compositions of the invention will need to meet certain criteria in order to be suitable for human or animal use and to meet the regulatory requirements. Thus, once the composition of the invention has been found to be suitable for animal administration, standard in-vivo and in vitro tests can be conducted to determine the information about the metabolism and pharmacokinetics of the compositions, including data on the drug-drug interactions where appropriate, which can be used to design human clinical trials. The present invention further contemplates that where toxicity is a factor, for example, in patients who cannot tolerate optimal or standard therapeutic dosages, or in cases where the patient's metabolism is compromised sub-optimal doses would be preferred.

### Determination of the Ability of the Plant Extracts to Inhibit Osteoclastic Bone Resorption:

Studies have determined that osteoclasts are largely derived from CD14 positive monocytes (J. Haemotol, 1999 July; 106(1):167-70). Clonal analysis of haematopoietic cells by surface phenotypes has been used to further identify osteoclast precursor by characterizing osteoclast like cells distinct from other haematopoietic progenitors (Lee et. al; 1992b Muguruma & Lee; 1988). Surface phenotype analysis has shown that human osteoclasts are derived from CD14 monocytes (Massey & Flanagan, 1999). CD 14 marker is strongly expressed on monocytes, the putative osteoclast precursor in peripheral blood and CD14 positive monocytes have been selected for osteoclastogenesis. (Clin. Sci (Lond) 2000, August, 99(2): 133-40).

Determination of the ability of the plant extracts to inhibit osteoclastic bone resorption has been undertaken wherein the potential extracts can be tested for their ability to inhibit these cell activity using a variety of techniques known in art, including, but not limited to, those described herein. In the context of the present invention, a plant extract that decreases the activity of the osteoclast cells by at least 40% in comparison to the control is considered capable of inhibiting osteoclastic bone resorption. Thus, in accordance with one embodiment of the invention there is provided a method of screening of plant extracts suitable for inclusion in the therapeutic compositions, the method comprising (a) providing one or more plant extract isolated with a specific solvent (b) analyzing the one or more extract for their inhibitory activity on bone resorption (c) selecting the extracts that decrease the activity of bone resorption by at least 40%, as plant extracts suitable for inclusion in the therapeutic compositions.

One skilled in art would appreciate that there are a variety of methods and techniques for measuring qualitatively and/or quantitatively the ability of the plant extract to have an inhibitory effect on osteoclastic bone resorption. For example there are currently several assays to measure bone resorption. Organ cultures rely upon the release in vitro of Ca from long bones or calvaria from newborn mice or rats (Raisz, 1963; van der Pluijm et al. 1994; Most et al. 1995). The bones are pre-labeled by incorporation into the pups of 45Ca or [3H] proline injected into the pregnant mother. The release of the isotopes from the cultured bones is measured at the end of the experiment, which can be continued for at least 5 days. This assay has been used frequently, but the cell composition of explants is extremely heterogeneous. Bone particle-based assays: Bone particles, obtained by grinding of 45Ca− or [3H] proline-labeled bones, are cultured with osteoclasts. 45Ca or [3H] release is measured at the end of the cultures (Oreffo et al. 1988). Bone slice assay. This widely used assay is based on the observation that isolated osteoclasts make resorption pits on slices of devitalised dentine or bone (Boyde et al. 1984) and that these pits resemble Howship's lacunae.

Various formats may be used if the potential extracts are to be tested against a specific set of cultured osteoclast cells. The assays may be adapted in order to facilitate the simultaneous testing of many potential extracts. Such techniques are being constantly developed and the use of such techniques to identify the potential extract activity is considered to be within the scope of the present invention.

### Determination of the Ability of the Plant Extracts Isolated to Promote Osteoblastic Bone Formation:

The many and varied osteoblast culture sywhole plants that have been developed include cultures containing osteoblast or osteoblast-like cells from different species, bones of different ages, and a variety of anatomical sites and pathological states. Sywhole plants have also been developed for specific cell populations, such as osteoprogenitor cells and osteocytes. Several recent articles have also discussed various osteoblast cell culture models and provide some critical commentaries about their use (Marie, 1994; Rodan et al. 1994; Gundle & Beresford, 1995, Parfitt, 1995; Roby, 1995). In the present invention MC3T3 cell line was used. These cells are murine osteogenic mesenchymal precursor cells, which can be differentiated into osteoblasts by ascorbic acid and beta-gylcerol phosphate.

Determination of the ability of the plant extracts to promote osteoblastic bone formation has been undertaken wherein the potential extracts can be tested for their ability to promote bone formation using a variety of techniques known in art, including, but not limited to, those described herein. In the context of the present invention, a plant extract that increases activity of the osteoblast cells. Thus, in accordance with one embodiment of the invention there is provided a method of screening of plant extracts suitable for inclusion in the therapeutic compositions, the method comprising (a) providing one or more plant extract isolated with a specific solvent (b) analyzing the one or more extract for their activity on bone formation (c) selecting the extracts that promote the activity of bone formation.

One skilled in art would appreciate that there are a variety of methods and techniques for measuring qualitatively and/or quantitatively the ability of plant extracts to have an effect on osteoblastic bone formation activity. Collagen and DNA synthesis, calcification and bone morphology can be tested in order to assay bone formation in culture. DNA synthesis may be measured by labeling bones with methyl [3H] thymidine for their last 2 h in culture (Gronowicz et al. 1994). The DNA content can be measured by fluorimetry (Labarca & Paigen, 1980). The measurement of Ca in cultured bone is an important indicator of bone formation in vitro. A calorimetric assay with o-cresolphthalein is commonly used to measure calcification in TCA extracts of cultured bones (Gronowicz et al. 1989). Calcein, a fluorescent dye that stains calcium phosphate deposits (Hock et al. 1968), can be used to measure calcification in mineralising cell cultures. Calcification, however, can be increased by bone damage or death (Ramp & Neuman, 1971). Therefore, the bone should also be checked by histological examination. Several histological methods can be used to assess bone morphology (Malluche & Faugere, 1986).

One of the most frequently assayed biochemical markers is alkaline phosphatase, which is simple to measure biochemically. (Sodek & Berkman, 1987). Its expression pattern in osteoblasts (Doty & Schofield, 1976; Stein et al. 1996) and its involvement in mineralisation have been extensively documented (Wuthier & Register, 1984). However, many cell types in bone or marrow stroma contain alkaline phosphatase, such as hypertrophic chondrocytes (Wuthier & Register, 1984) and adipocytes (Beresford et al. 1993), while fibroblastic cells also have a low concentration of this enzyme. The alkaline phosphatase activity can provide a good indicator of osteoblast cells if the cartilage and marrow are removed by dissection. The present invention evaluates the bone formation that has occurred through Alkaline phosphatase assay and the proliferation that has occurred through Alamar blue assay.

Various other in-vitro osteogenic potential assays are being constantly developed and the use of such techniques to identify the potential extract activity are considered to be within the scope of the present invention.

Various cell lines can be used in the above assays. Examples of suitable cell lines such as ST2 (mature monocytes and macrophages capable of differentiating into osteoclasts, MLC-6 (osteoclast like cell line derived from mouse, MC3T3-E1 (mouse calvaria, Sudo et al 1983), MBA-15 (Clonal marrow stromal cell line) and the like. Osteoblast cell lines include 2T3 (osteoblast cell line), AHTO, HOBIT cell lines and the like can be used for the cell based assays. These cell lines can be obtained from ATCC or various other commercial sources. The invention premeditates the use of such suitable osteoclast and osteoblast cell lines for conducting the cell based assays.

Also encompassed within the ambit of the invention is a pharmaceutical formulation suitable for use in the treatment of Osteoporosis, comprising at least one extract as isolated from a Cissus quadrangularis, in admixture with a pharmaceutically acceptable carrier. Preferably, the at least one extract is selected from those listed in Table 1. Naturally, the skilled addressee will appreciate that such compositions may comprise of two or more plant extracts of the invention in any concentration, which is capable of giving rise to a therapeutic effect. Thus, therapeutic compositions can comprise plant extracts of Cissus substantially devoid of undesirable contaminating compounds. The plant extracts may have, for procedure, undergone a number of solvent extraction steps substantially to separate out undesirable components from desirable components such as those alluded to in the procedures and aforementioned tables.

The invention thus further provides a method for the treatment of Osteoporosis or conditions, which are characterized by increased bone resorption in mammals, including humans, which comprises the use of a clinically useful amount of an extract selected from those listed in Table 1, in a pharmaceutically useful form, once or several times a day or in any other appropriate schedule for example, orally, or intravenously or by delivery to the lungs in a dry or “wet” spray.

The amount of compound of extract required to be effective in the treatment Osteoporosis or conditions, which are characterized by increased bone resorption will, of course, vary with the disease being treated and is ultimately at the discretion of the medical or veterinary practitioner. The factors to be considered include the condition being treated, the route of administration, and nature of the formulation, the mammal's body weight, surface area, age and general condition and the particular compound to be administered. A suitable effective dose of an extract of the invention generally lies in the range of about 500 mg twice daily.

Whilst it is possible for the active extract to be administered alone, it is preferred to present the active extract in a pharmaceutical formulation. Formulations of the present invention, for medical use, comprise an extract of the invention together with one or more pharmaceutically acceptable carriers and optionally other therapeutic ingredients. The carrier(s) should be pharmaceutically acceptable in the sense of being compatible with the other ingredients of the formulation and substantially non-deleterious to the recipient thereof.

The present invention, therefore, further provides a pharmaceutical formulation comprising at least one extract selected from those listed in table 1, together with a pharmaceutically acceptable carrier therefore.

Naturally, the skilled addressee will appreciate that any pharmaceutical formulation comprising an active extract of the invention can include at least one active extract purified from an extract derived from a Cissus species. Thus a pharmaceutical formulation may contain more than one active extract derived from two or more Cissus species.

There is also provided a method for the preparation of a pharmaceutical formulation comprising bringing into association an extract of the invention, and a pharmaceutically acceptable carrier therefore. Formulations according to the present invention include those suitable for oral or intravenous administration.

Intravenous formulations including at least one extract of the invention and may also be administered in the form of suitable liposomal or niosomal preparations or other suitable delivery vehicle.

Emulgents and emulsion stabilizers suitable for use in the formulation of the present invention include Tween 60, Span 80, cetostearyl alcohol, myristyl alcohol, glycerol mono-stearate and sodium laury sulphate.

The formulations may conveniently be presented in unit dosage form and may be prepared by any of the methods well known in the art of pharmacy. All methods include the step of bringing the active extracts(s) into association with a carrier, which constitutes one or more accessory ingredients. In general, the formulations are prepared by uniformly and intimately bringing the active extract(s) into association with a liquid carrier or a finely divided solid carrier or both and then, if necessary, shaping the product into desired formulations.

Formulations of the present invention suitable for oral administration may be presented as discrete units such as capsules, sachets, tablets, lozenges, comprising the active ingredient in a flavoured based, usually sucrose and acacia or tragacanth; pastilles comprising the active ingredient in an inert base such as gelatin and glycerin, or sucrose and acacia; and mouth-washes comprising the active ingredient in a suitable liquid carrier. Each formulation generally contains a predetermined amount of the active extract; as a powder or granules; or a solution or suspension in an aqueous or non-aqueous liquid such as syrup, an elixir, an emulsion or draught and the like.

A tablet may be made by compression or moulding optionally with one or more accessory ingredients. Compressed tablets may be prepared by compressing an a suitable machine the active extract in a free-flowing form such as a powder or granules, optionally mixed with a binder, (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface active or dispersing agent. Moulded tablets may be made by moulding in a suitable machine a mixture of the powdered extract moistened with an inert liquid diluent. The tablets may optionally be coated or scored and may be formulated so as to provide slow or controlled release of the active ingredient therein using, for example, hydroxypropylmethylcellulose in varying proportions to provide the desired release profile.

A syrup may be made by adding the active extract to a concentrated, aqueous solution of a sugar, for example sucrose, to which may also be added any necessary ingredients. Such accessory ingredient(s) may include flavourings, an agent to retard crystallisation of the sugar or an agent to increase the solubility of any other ingredients, such as a polyhydric alcohol for example glycerol or sorbitol.

In addition to the aforementioned ingredients, the formulations of this invention may further include on or more accessory ingredients) selected from diluents, buffers, flavouring agents, binders, surface active agents, thickeners, lubricants, preservatives (including antioxidants) and the like.

Alternatively, the compositions are dietary supplements, food compositions or beverage compositions suitable for human or animal consumption.

In a further aspect of the invention there is provided a comestible, that is to say, a foodstuff comprising at least an extract of the inventions typically in dried form, such as in a lyophilized form selected from those listed in Table 1 herein. The skilled addressee will appreciate that such comestibles may contain more than one extract of the invention and may be used. Such foodstuffs may be used in a prophylactic manner and may contain further extracts having a similar function to the first added extract or further added extracts may be added that have a different prophylactic function. Thus a foodstuff could either comprise extracts that provide for a comestible having a single functional aspect, or a comestible may have a multi-functional prophylactic effect against two or more disease types. It is thought that a similar multi-functional role could also be assigned to pharmaceutical formulations comprising two or more extracts possessing dissimilar therapeutic or prophylactic properties designed either for prophylaxis or for the treatment of more than one disease(s) in a mammal, particularly in a human.

The type of foodstuff or comestible to which at least an extract of the invention may be added includes any processed food such as confectionaries, baked products including breads such as loafs, and flat breads such as pitta bread, naan bread and the like, cakes, snack foods such as muesli bars, compressed dried fruit bars, biscuits, dairy products such as yoghurts, milk and milk-based products such as custards, cream, cheese, butter and crème fraiche, simulated dairy food products such as margarine, olive oil-based spreads, and low fat cream substitutes such as Elmlea products, fruit and vegetable juices, aerated drinks, such as carbonated soft drinks and non-aerated drinks such as squashes, soya milk, rice milk and coconut milk and the like, pastas, noodles, vegetable, seed and nut oils, fruited oils such as sunflower oil, rapeseed oil, olive oil, walnut, hazelnut, and sesame seed oil and the like, and frozen confections such as ice creams, iced yoghurts and the like.

A suitable effective dose of an extract of the invention to be included in a comestible generally lies in the range 500 mg twice daily.

The invention will now be exemplified with reference to the following Procedures section and accompanying tables and Figures. It is to be understood that the procedures are not to be construed as limiting the scope of the invention in any way.

### Example 1

### Extraction of Cissus quadrangularis

Extraction of Cissus quadrangularis whole plant was carried out by direct extraction method, at 20-85° C. in reflux extractor apparatus followed by Lyophilizing the obtained extracts between 50 to 500 m Torr (optimum 110 m Torr) and between −30 to −80° C. temperature (optimum −60° C.). Various solvents used for extraction are 80% Ethanol, Hexane Acetone and water.

The detailed process is given below:

10-300 grams of powdered plant material was weighed into the round-bottomed extractor flask. 50-1000 ml of solvent was added in to the flask and placed on the mantle along with few (3-4) ceramic chips. The flask is fitted with a water-cooled condenser. The mantle was switched on and the desired temperature was set. During extraction the solvent boils in presence of plant material leading to the extraction of compounds from the plant material. This process is continuous as long as there is stable heat and water circulation. The extraction was continued for 4-6 hours, 4-5 cycles per hour. After four to six hours the mantle was switched off and the water flow was stopped. After cooling the plant material was removed by filtration through a cotton plug.

The extract was concentrated by fitting the flask containing the extract with the empty soxhlet extractor body that in turn was fitted tightly with the water-cooled condenser. Continuous water flow was maintained and the flask was heated till the solvent from the flask was distilled and collected in the extractor body up to a level. (One inch below the inlet). The temperature was reduced to avoid charring as the volume of the solvent reduced in the flask. The distilled solvent collected in the extractor was transferred to the solvent bottles and label appropriately. The process was continued till only very little solvent was left in the flask and no charring had occurred. The extract in the flask were swirled and were dried under vacuum.

Storage and labeling of the extract was done to obtain the Extract ID. The Extract ID contains the first six letters of the plant code followed by the part of the plant used. This is followed by type of extraction, extraction temperature, solvent and the percentage of the solvent used. Which is followed by the date, month and year of extraction for the identification of the three extracts, which are the subject matter of the invention.

For Example: AGT036_Wp_Dr Rf(65)04(80)_{—}14_{—}07_{—}05

AGT036→Name of the Plant: Cissus quadrangularis
Wp→Part of the plant: Whole plant
DrRf→Type of extraction Direct reflux

### 65→Extraction Temperature 65° C.

04→Solvent code Ethanol
00→Percentage of the solvent 80%

14_{—}07_{—}05→ 14^{th} Jul. 2005

### Example 1

200 grams of powdered plant material was weighed into the round-bottomed extractor flask. 1000 ml of solvent was added in to the flask along with few (3-4) ceramic chips and placed on the mantle. The flask is fitted with a water-cooled condenser. The mantle was switched on and the temperature was set to 65° C. The extraction was continued for 5 hours, 4-5 cycles per hour. After 5 hours the mantle was switched off and the water flow was stopped. After cooling the plant material was removed by filtration through a cotton plug followed by Lyophilizing the obtained extracts between 50 to 500 m Torr (optimum 110 m Torr) and between −30 to −80° C. temperature (optimum −60° C.).

TABLE 1
Extracts of the whole plants of Cissus quadrangularis
with different solvents

| Sr. | | | % | Screening |
|---|---|---|---|---|
| No. | Solvent | Extract Name | Yield | name |
| 1 | 80% ethanol | AGT036_WP_DrRf(65)04(80)_14_07_05 | 1.65 | AN01 |
| 2 | Hexane | AGT036_WP_DrRf(65)05(00)_14_07_05 | 2.9 | AN02 |
| 3 | Acetone | AGT036_WP_DrRf(65)01(00)_19_07_05 | 5.7 | AN03 |
| 4 | Water | AGT036_WP_DrRf(65)08(00)_28_07_05 | 17.31 | AN19 |

### Example 2

100 grams of powdered plant material was weighed into the conical flask. 500 ml of solvent was added in to the flask and the flask was kept on a thermostat shaker set at 25° C. The extraction was continued for 6 hours, 4-5 cycles per hour. After six hours the plant material was removed by filtration through a cotton plug followed by Lyophilizing the obtained extracts between 50 to 500 m Torr (optimum 110 m Torr) and between −30 to −80° C. temperature (optimum −60° C.).

TABLE 2
Extracts of the whole plants of Cissus quadrangularis
with different solvents

| Sr. | | | % |
|---|---|---|---|
| No. | Solvent | Extract Name | Yield |
| 1 | 80% ethanol | AGT036_WP_DrRf(25)04(80)_14_07_05 | 0.71 |
| 2 | Hexane | AGT036_WP_DrRf(25)05(00)_14_07_05 | 1.9 |
| 3 | Acetone | AGT036_WP_DrRf(25)01(00)_19_07_05 | 3.8 |
| 4 | Water | AGT036_WP_DrRf(25)08(00)_28_07_05 | 8.31 |

### Example 3

20 grams of powdered plant material was weighed into the round-bottomed extractor flask. 100 ml of solvent was added in to the flask along with few (3-4) ceramic chips and placed on the mantle. The flask is fitted with a water-cooled condenser. The mantle was switched on and the temperature was set to 85° C. The extraction was continued for 6 hours, 4-5 cycles per hour. After six hours the mantle was switched off and the water flow was stopped. After cooling the plant material was removed by filtration through a cotton plug followed by Lyophilizing the obtained extracts between 50 to 500 m Torr (optimum 110 m Torr) and between −30 to −80° C. temperature (optimum −60° C.).

TABLE 3
Extracts of the whole plants of Cissus quadrangularis
with different solvents

| Sr. | | | % |
|---|---|---|---|
| No. | Solvent | Extract Name | Yield |
| 1 | 80% ethanol | AGT036_WP_DrRf(85)04(80)_14_07_05 | 0.63 |
| 2 | Hexane | AGT036_WP_DrRf(85)05(00)_14_07_05 | 1.6 |
| 3 | Acetone | AGT036_WP_DrRf(85)01(00)_19_07_05 | 3.1 |
| 4 | Water | AGT036_WP_DrRf(85)08(00)_28_07_05 | 7.62 |

### Preparation of Stock Solutions:

50 mg of each extract were weighed and dissolved in DMSO.

Concentrations of each extracts are given in Table 4.

TABLE 2
Concentration of the stock solutions of
the extracts of Cissus quadrangularis

| Screening | Extract | | |
|---|---|---|---|
| name | number | 50 mg extract dissolved in: | Concentration |
| AN01 | 1 | 300 μl DMSO | 167 μg/μl |
| AN02 | 2 | 600 μl DMSO | 83 μg/μl |
| AN03 | 3 | 600 μl DMSO | 83 μg/μl |
| AN19 | 19 | 600 μl DMSO | 83 μg/μl |

### Example 4

### Effect of the Extracts of Cissus quadrangularis on MC3T3 Cells (Toxicity Testing)

The plant extracts were tested in three concentrations to examine the toxicity of the extracts to MC3T3 cells. The stock solutions of the extract were diluted 1:100, 1:1000 and 1:10000 in the medium for the cells. The concentrations have been chosen so that the concentration of DMSO used for making the stock solutions is not higher than 1%.

MC3T3 cells were seeded at a density of 20000 cells per well in 96 well plate. The cells were cultured for one day before the plant extracts were added in the three dilutions. The cells were after the addition of the extracts cultured for three days. At the end of the culture period the cell viability was measured by the AlamarBlue assay.

Plant extract AN02 and AN03 were toxic at the dilution 1:100, but not at dilution 1:1000 and 1:10,000, while extract AN01 and AN19 were not toxic at either of the dilutions. Therefore, dilution 1:1000 and 1:10,000 were used for the hit screening in the bone resorption assay and in the bone formation assay.

### Example 5

### Determination of the Bio-Therapeutic Potential of the Extracts of the Whole Plants of Cissus quadrangularis

### (a) Bone Resorption

### Principle

Collagen type I makes up >90% of the organic matrix of bone. Collagen type I is composed of linked type I collagen molecules, with the amino- and carboxy-terminal regions of a type I collagen molecule linked to the helical region of adjacent collagen molecules through pyridinium cross-links, deoxypyridinoline (Dpd) and pyridinoline (Pyd).

The breakdown of collagen type I, which is mediated by osteoclast-derived acid proteases, leads to the release of free and peptide-bound metabolites of the type I collagen molecule. Fragments released from the amino- and carboxy-terminal regions of collagen type I are termed the N-terminal telopeptide (NTX) and the C-terminal telopeptide (CTX), respectively. These telopeptides consist of short peptide sequences from the helical domain of a type I molecule and the telopeptide region of an adjacent molecule, joined by a pyridinium cross-link (either Pyd or Dpd). A proportion of the telopeptides released from bone are excreted unchanged in urine, and the remainder are metabolized by the liver and kidneys. Commercial assays have been developed for 3 forms of collagen type I telopeptide: NTX, CTX, and ICTP (a slight variant of CTX).

During renewal of the skeleton bone matrix is degraded and consequently fragments of type I collagen is released into circulation. The resorption process can be studied in vitro by culturing bone cells on devitalized slices of bone or dentin.

### Method

The osteoclasts were isolated as CD14 positive monocytes and differentiated into osteoclasts for 10-12 days. Hereafter, the osteoclasts were lifted by trypsin, counted and seeded on bovine bone slices at a density of 40000 cells per bone slice. The osteoclasts were seeded on the bone slices for one day before the plant extracts were added. The osteoclasts were hereby allowed to attach and initiate resorption.

The plant extracts were added in two concentrations 1:10,000 and 1:100,000 dilutions because the toxicity study in the MC3T3 cells had shown that these concentrations were not toxic to the cells.

Bafilomycin is a V-ATPase blocker and it inhibits the osteoclastic bone resorption approximately 80% compared to DMSO (the negative control will be set to 100% on the chart.

The bone resorption experiment was ended after 5 days of incubation and the cell viability was measured by the use of the AlamarBlue assay. Bone resorption was evaluated as the determination of the CTX release.

Plant extract AN01, which is Cissus quadrangularis extract made with 80% ethanol, has an inhibitory effect on the bone resorption at dilution 1:1000. AN01 inhibit bone resorption ˜90% compared to the control, which is not due to toxicity.

Plant extract AN02, which is Cissus quadrangularis extract made with hexane, inhibit resorption at both dilution 1:1000 and 1:10,000. At dilution 1:1000 inhibits AN01 bone resorption ˜85% compared to the control. Some of the effect is due to toxicity, because dilution 1:1000 are toxic to ˜50% compared to the control.

Plant extract AN03, which is Cissus quadrangularis extract made with acetone, inhibit bone resorption at 1:1000, which seems to be due to toxicity. There is a small inhibition of the resorption at 1:10,000, which does not seem to be because of toxicity. At 1:10,000 AN03 inhibit bone resorption ˜40% compared to the control.

Plant extract AN19, which is Cissus quadrangularis extract made with water, has no effect on bone resorption.

Plant extract AN01, AN02 and AN03 were selected for further test in bone resorption lead characterisation.

The result for the lead characterization of plant extract AN01, AN02 and AN03 gave the same result as the hit screening. The inhibitor effect of the extracts on bone resorption was reproduced by the lead characterization. Extract AN01 and AN03 inhibit resorption totally at dilution 1:1000, while extract AN02 inhibit resorption ˜80% compared to the control. All three extracts are toxic to a small extend at this dilution. At dilution 1:100 all the extracts are toxic to the osteoclasts, which is also seen in low TRAP activity the lower dilution. The TRAP activity decreases due to the dose response of the extract.

### Example 6

### Bone Formation Studies

MC3T3 cell lines were used for the bone formation studies. These cells are murine osteogenic mesenchymal precursor cells, which can be differentiated into osteoclasts by ascorbic acid and beta-glycerol phosphate.

The osteoblasts (MC3T3 cells) were seeded at a density of 75000 cells per well in 24 plates. The cells were culture for one day before the plant extracts were added. Then the osteoblasts were cultured for 14 days in medium supplemented with ascorbic acid and beta-glycerol phosphate (AA and bG) and in the presence of the plant extracts (1:10,000 and 1:100,000 dilution)

On each plate were a negative control and a positive control added. Medium added without ascorbic acid and beta-glycerol phosphate (without AA and bG) was used as a negative control and this condition does not promoted differentiation of osteoblasts. A positive control was 30 ng/ml BMP-2 used (BMP-2=bone morphogenic protein −2), which induces the differentiation of the osteoblasts.

For the bone formation experiment BMP-2 is used as a positive control, while the treatment without ascorbic avid and beta-glycerol phosphate (without AA and bG) is the negative control. BMP-2 induces the MC3T3. precursor cells to differentiate into osteoblasts and the ALP activity is induced compared to the negative control.

For the bone formation experiment is BMP-2 used as a positive control, while treatment without ascorbic acid and β-glycerol phosphate (without aa and bG) is the negative control. BMP-2 induces the MC3T3 precursor cells to differentiate into osteoblasts and the ALP activity is induced compared to the negative control. From measurement of ALP activity it can be concluded that plant extract AN03 has a positive osteogenic effect on the MC3T3 cells. The ALP activity is higher for AN03 compared to the negative control and it induces ALP activity to a higher level than BMP-2. Furthermore, AN03 was not toxic to the cells at the used dilutions.

1. A method for treating osteoporosis or other related disorders such as bone loss, bone fracture, glucocorticoid induced osteoporosis, Pagets disease, osteoarthritis, peri-prosthetic osteolysis, cartilage degeneration, osteogenesis imperfecta and the like, which comprises administering to the said mammal an effective non-toxic amount of an extract derived from Cissus plant species.

2. A method of treating osteoporosis or other related disorders such as bone loss, bone fracture, glucocorticoid induced osteoporosis, Pagets disease, osteoarthritis, peri-prosthetic osteolysis, cartilage degeneration, osteogenesis imperfecta according to claim 1, which comprises administering to the said mammal an effective non-toxic amount of at least an extract from Cissus quadrangularis selected from the group consisting of AGT036_Wp_DrRf(65)04(80)_{—}14_{—}07_{—}05, AGT036_WP_DrRf(65)05(00)_{—}14_{—}07_{—}05, AGT036_WP_DrRf(65)01(00)_{—}19_{—}07_{—}05 & AGT036_WP_DrRf(65), 08(00)_{—}28_{—}07_{—}05 or a combination of two or more extracts thereof.

3. A method according to claim 1, wherein the said treatment is a prophylactic treatment.

4. (canceled)

5. (canceled)

6. (canceled)

7. (canceled)

8. A comestible comprising at least an extract from Cissus plant species.

9. A comestible according to claim 8, comprising at least an extract from Cissus quadrangularis selected from the group consisting of AGT036_Wp_Dr Rf(65)04(80)_{—}14_{—}07_{—}05, AGT036_WP_DrRf(65)05(00)_{—}14_{—}07_{—}05 AGT036_WP_DrRf(65)01(00)_{—}19_{—}07_{—}05 & AGT036_WP_DrRf(65) 08(00)_{—}28_{—}07_{—}05 or a combination of two or more extracts thereof.

10. A comestible according to claim 8 comprising at least an extract for use in the prophylaxis of osteoporosis or other related disorders such as bone loss, bone fracture, glucocorticoid induced osteoporosis, Pagets disease, osteoarthritis, peri-prosthetic osteolysis, cartilage degeneration, osteogenesis imperfecta and the like.

11. (canceled)

12. (canceled)

13. A method of producing a Cissus plant extract having bone resorption and bone formation properties comprising the following steps:
(a) Pulverizing selected plant material in an amount of 20-300 gr to a powder;
(b) Subjecting the powdered plant material to solvent extraction using 100-1000 ml;
(c) Lyophilizing the obtained extracts at a pressure of between 50 to 500 m Torr and a temperature of between −30 to −80° C.

14. A Method of producing a Cissus Plant extract as claimed in claim 13, wherein the said step of lyophilisation is done optimally at a temperature of −60° C.

15. A Method of producing a Cissus Plant extract as claimed in claim 13, wherein the said step of lyophilisation is done optimally at a pressure of 110 m Torr.

16. The medicinal plant extract derived from Cissus plant species produced according to claim 13.
